(19) United States
(12) Patent Application Publication
(10) Pub. No.: US 2022/0305064 A1
(43) Pub. Date: Sep. 29, 2022

(54) LACTOBACILLUS COMPOSITIONS AND USES THEREOF

(71) Applicant: Probi AB, Lund (SE)

(72) Inventors: Magnus Hillman, Södra Sandby (SE); Siv Ahrné, Blentarp (SE); Gunilla Önning, Höör (SE)

(21) Appl. No.: 17/616,968

(22) PCT Filed: Jun. 5, 2020
(86) PCT No.: PCT/EP2020/065620

§ 371 Date: Dec. 6, 2021

(30) Foreign Application Priority Data
Jun. 7, 2019 (GB) 1908154.6

(51) Int. Cl. A61K 35/747; A61P 25/22

(52) U.S. Cl. CPC A61K 35/747; A61P 25/22

(57) ABSTRACT

The invention relates to at least one strain of Lactobacillus for use in a method of reducing and/or preventing at least one deleterious effect of acute psychosocial stress in a human, wherein the method comprises treatment by administration of an effective dose of the at least one strain of. The at least one deleterious effect may be an elevated level of soluble fractalkine and may be in combination with at least one further deleterious effect of acute psychosocial stress.

### TECHNICAL FIELD OF THE INVENTION

The present invention relates to at least one strain of Lactobacillus for use in a method of reducing and/or preventing at least one deleterious effect of acute psychosocial stress in a human, wherein the method comprises treatment by administration of an effective dose of the at least one strain of Lactobacillus, preferably wherein the at least one deleterious effect is an elevated level of soluble fractalkine, optionally in combination with at least one further deleterious effect of acute psychosocial stress.

### BACKGROUND OF THE INVENTION

Stress is an organism's response to a stressor such as an environmental condition. Stress includes the body's method of reacting to a condition such as a threat, challenge or physical or psychological barrier. Such conditions, threats, challenges or barriers are known as stressors. The autonomic nervous system and hypothalamic-pituitary-adrenal (HPA) axis are two major systems that respond to stress.

The sympathoadrenal medullary (SAM) axis may activate the fight-or-flight response through the sympathetic nervous system, while the parasympathetic nervous system returns the body to homeostasis. The HPA axis regulates the release of cortisol, which influences many bodily functions such as metabolic, psychological and immunological functions. The SAM and the HPA axes are regulated by several brain regions, including the limbic system, prefrontal cortex, amygdala, hypothalamus and stria terminalis.

Acute stress over-activates the immune system, leading to an imbalance between inflammation and anti-inflammation. Through disturbing the balance of immune system, stress induces inflammation peripherally and centrally. This imbalance leads to diversified stress-related diseases like cardiovascular diseases, neurodegenerative diseases and cancer (Liu et al, 2017, Front Hum Neurosci; 11: 316).

Stress also increases the response of the gastrointestinal system to inflammation and may reactivate previous inflammation and accelerate the inflammation process (Yaribeygi et al, 2017, EXCLI J. 16: 1057-1072). For example, chronic stress may increase the risk of inflammatory bowel disease (IBD), which may be exacerbated by acute psychosocial stress. Irritable bowel syndrome, which in part can have an inflammatory origin, is also related to stress.

Thus, there is a need within the art to find effective methods for reducing and/or preventing the deleterious effects of acute psychosocial stress in humans, particularly in individuals who also suffer from chronic stress.

### STATEMENT OF THE INVENTION

The invention provides at least one strain of Lactobacillus for use in a method of reducing and/or preventing at least one deleterious effect of acute psychosocial stress in a human, wherein the method comprises treatment by administration of an effective dose of the at least one strain.

In accordance with the invention, administration of the at least one strain of Lactobacillus preferably reduces and/or prevents at least one deleterious effect of acute psychosocial stress in a human compared to the at least one deleterious effect of acute psychosocial stress in the human in the absence of the at least one strain.

In a first aspect, the administration of the at least one strain of Lactobacillus reduces and/or prevents elevated levels of soluble fractalkine as the deleterious effect of acute psychosocial stress.

In a second aspect, the administration of the at least one strain of Lactobacillus reduces and/or prevents elevated levels of soluble fractalkine as a deleterious effect on acute psychosocial stress in combination with reducing or preventing at least one further deleterious effect of acute psychosocial stress. The at least one further deleterious effect may be selected from a biochemical indicator and/or a physiological indicator. The at least one further deleterious effect may be an elevated level of soluble CD163.

In a third aspect, the administration of the at least one strain of Lactobacillus reduces and/or prevents elevated levels of soluble CD163 as a deleterious effect of acute psychosocial stress. The administration may reduce or prevent one or more further deleterious effects of acute psychosocial stress.

In a fourth aspect, the administration of the at least one strain of Lactobacillus reduces and/or prevents at least one deleterious effect of psychosocial stress, wherein the deleterious effect is a physiological indicator. The physiological indicator may be selected from increased pulse, increased heart rate, increased high frequency heart rate variability, increased gut permeability, and adverse gut function, such as increased abdominal pain, flatulence and/or bloating.

By “use in reducing and/or preventing” we include the meaning of a use which gives rise to an effect in a subject of preventing, delaying, protecting against, reducing the severity of and/or removing, one or more effects, symptoms and/or other markers associated with a disorder, disease or condition.

By “prevent”, “prevention” or “preventing” we include the meaning that the event, effect or condition being prevented is protected against, delayed, reduced (e.g. reduced in severity), blocked from occurring, or made to cease. Such prevention typically takes place before the event occurs or the effect or condition is manifest, but it will be appreciated that it can also mean to prevent further occurrence of the same kind of event. It will also be appreciated that such terms may include the meaning that an event or condition is maintained in the current state without becoming worse or developing further.

For example, a measure of a biochemical indicator of stress (e.g. soluble fractalkine) associated with, or preceding an episode of acute psychosocial stress may be reduced by at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98% or at least 99% following administration of the at least one strain (or of a composition comprising the at least one strain) according to the invention compared to without administration of the at least one strain, or compared to administration of a corresponding composition lacking the at least one strain.

By “psychosocial stress” we include the meaning of stress that is induced by situations of social threat, which can include social evaluation, social exclusion and ‘achievement’ situations claiming goal-directed performance (see Kogler et al, 2015, Neuroimage 119:235-251, the entire contents of which are incorporated herein by reference). Psychosocial stress can arise from situations in which gratification of the need to be affiliated with others, and/or the need to maintain the social-self, is threatened. An ‘achievement situation’ is a situation in which an individual believes that his or her performance will be evaluated. Hence, psychosocial stress includes stress induced by situations in which there is a threat that an individual's performance will be judged negatively by others.

It will be appreciated that the term “psychosocial stress” includes the effect(s) induced by particular psychosocial stressors. Hence, psychosocial stressors include the potential for negative judgment of one's performance by others, particularly where social exclusion or a lack of achievement could result from that judgment.

It will be appreciated that psychosocial stress is intended to exclude physiological stress. By “physiological stress” we include the meaning of stress that is associated with potential damage of body tissue and bodily threat, such as pain, hunger, oxidative stress, etc. (see Kogler et al, 2015, supra). It will also be appreciated that psychosocial stress is intended to exclude stress induced by physical activity or exercise, such as as described in WO 00/70972, and any stress induced by infection or allergy. Physiological stress triggers the ‘fight-or-flight’ reaction, whereas during psychosocial stress, attention is shifted towards emotion regulation and goal-directed behaviour, with a reduction in reward processing (Kogler et al, 2015, supra).

By “acute” in the context of “acute psychosocial stress” we include the meaning that the presence of the stressor, and/or the stress response to the stressor, is time-limited. Hence, an acute stressor typically involves a short-term challenge. For example, an acute stressor (and/or the stress response to that stressor) is typically present for less than one day, such as no more than 12, 11, 10, 9, 8, 7, or preferably less than 6, 5, 4, 3, 2 or 1 hour(s). It will be appreciated that more than one episode of acute psychosocial stress could take place in a day and also that the same or a similar acute stressor could result in more than one episode of acute psychosocial stress within a short period (e.g. within a 24- to 72-hour period) and still be considered as causing acute stress.

Examples of acute psychosocial stressors (or stimuli that cause acute psychosocial stress) include, but are not limited to the following situations: being interviewed; giving an oral presentation or talk; undergoing an examination; making an important telephone call; participating in an important meeting; having to work up to a deadline (or close to an imminent deadline); being bullied; receiving unexpected serious news such as illness, bereavement or unemployment.

It will be appreciated that the term “acute” is intended to generally exclude the meaning of the term “chronic”. By “chronic” we include the meaning of a persistent state. It will be appreciated that a persistent state can include fluctuations and does not only include a static state. In contrast to the limited time frame of “acute”, for a situation to be “chronic” typically requires repetition, or persistence, over several days, preferably over more than 1, 2, 3, 4, 5 or 6 weeks, or more than 1, 2, 3, 4, 5, 6, 7, 8, 9, 10, 11, or 12 months.

By “chronic stress” we include the meaning of a persistent state of stress. Chronic stress typically includes the response to emotional pressure suffered for a prolonged period of time in which an individual perceives they have little or no control. Examples of a state of chronic stress are those following serious life events, such as a period of bereavement, a period of unemployment, loneliness, or ongoing marital problems.

It will also be appreciated that an acutely stressful event could precede or lead to a period of chronic stress. For example, the acute stressor could be the receipt of unexpected serious news (e.g. illness, bereavement, or unemployment), and the chronic stressor could be the persistence of the negative situation that was the subject of the news (e.g. the illness, bereavement, or unemployment).

By “effect of acute psychosocial stress” we include the meaning of a physical measure that directly or indirectly results from the presence of the acute psychosocial stressor. By “deleterious” we include the meaning of causing harm or damage. Hence, in relation to a “deleterious effect of acute psychosocial stress”, we include the meaning that the acute psychosocial stress can cause, worsen or exacerbate a particular indicator that causes harm or damage to the individual.

Preferably, the deleterious effect of acute psychosocial stress is selected from one or more of a biochemical and/or a physiological indicator of stress. It will be appreciated that the deleterious effect of acute psychosocial stress may be manifest (e.g. measurable) before, during, or after the episode of acute psychosocial stress. Preferably, the deleterious effect of acute psychosocial stress is measurable during and/or within one hour after the episode of acute psychosocial stress.

Examples of biochemical indicators of stress include elevated levels of cytokines, especially inflammatory cytokines and/or zonulin and/or cortisol and/or soluble CD163. Preferably the inflammatory cytokines are one or more of soluble fractalkine (chemokine [C-X3-C motif] ligand 1, encoded in humans by the CX3CL1 gene). C-reactive protein (CRP, encoded in humans by the CRP gene), interferon gamma (IFNγ, encoded in humans by the IFNG gene), interleukin 10 (IL-10 or human cytokine synthesis inhibitory factor [CSID], encoded in humans by the IL10 gene), interleukin 1 beta (IL-1β, encoded in humans by the IL1B gene), interleukin 6 (IL-6, encoded in humans by the IL6 gene), interleukin 8 (IL-8 or chemokine [C-X-C motif] ligand 8, CXCL8, encoded in humans by the CXCL8 gene), or tumor necrosis factor alpha (TNFα, cachexin or cachectin, encoded in humans by the TNFA gene). Zonulin, also known as haptoglobin 2 precursor (uncleaved form of allele alpha-2 [2-2]) is a protein encoded by the HP gene in humans (see Fasano, 2011, Physiol Rev, 91(1):151-175, the entire contents of which are incorporated herein by reference) and modulates the permeability of tight junctions between cells of the wall of the digestive tract. Zonulin is considered to be the mammalian analogue of zonula occludens toxin secreted by Vibrio cholerae, and hence, zonulin has been implicated in the pathogenesis of coeliac disease and diabetes mellitus type 1. Cortisol, also known as hydrocortisone, is a steroid hormone in the glucocorticoid class of hormone, and is mainly produced in the adrenal cortex within the adrenal gland, but there is also evidence of local synthesis in the intestine (see Taves et al, 2011, Am J Physiol Endocrinal Metab, 301(1):E11-E24, the entire contents of which are incorporated herein by reference). CD163 (encoded in humans by the CD163 gene) is a scavenger receptor belonging to the scavenger receptor cysteine rich family type B. Membrane-bound CD163 has a 1048 amino acid residue extracellular domain, a single transmembrane segment and a cytoplasmic tail with several splice variants. Soluble CD163 (sCD163) is generated by ectodomain shedding of the membrane-bound receptor as a result of enzymatic cleavage by ADAM17, also known as tumor necrosis factor-alpha converting enzyme (TACE). sCD163 is found in plasma and cerebrospinal fluid. sCD163 is upregulated in a large range of inflammatory diseases including liver cirrhosis, type 2 diabetes, macrophage activation syndrome, Gaucher's disease, sepsis, HIV infection, rheumatoid arthritis and Hodgkin Lymphoma. sCD163 is also upregulated in cerebrospinal fluid after subarachnoid haemorrhage.

Biochemical indicators of stress can be determined by any suitable measure known in the art. Preferably, biochemical indicators of stress are measured in plasma or serum. For example, levels of cytokines (including chemokines), peptides or proteins may be determined by enzyme-linked immunosorbent assay (ELISA) with antibodies specific to the cytokine to be measured, or by a multiplex kit from MesoScale Discovery, Rockville, Md., US. Measurement techniques for hormones, such as cortisol, include immunoassay (e.g. ELISA) and liquid chromatography with tandem mass spectrometry (LC-MS/MS). It will also be appreciated that biochemical indicators of stress may be measured in other biological samples, e.g. urine, faeces, saliva, breath, tears, lymph fluid, cerebrospinal fluid, synovial fluid, or tissue biopsy.

Examples of physiological indicators of stress include increased pulse, increased heart rate, increased high frequency heart rate variability, increased gut permeability, and adverse gut function, such as increased abdominal pain, flatulence and/or bloating.

Physiological indicators of stress can be determined by any suitable measure known in the art. For example, measures of heart rate may be determined by electrocardiography and measures of gut function may be determined by questionnaire with visual analogue scale.

In a most preferred embodiment, the biochemical indicator of stress is an elevated level of soluble fractalkine, preferably an elevated level of soluble fractalkine in plasma.

Hence, in a preferred embodiment according to the invention, the at least one strain of Lactobacillus is for use in a method of reducing and/or preventing an elevated level of soluble fractalkine in plasma as a deleterious effect of acute psychosocial stress in a human, wherein the method comprises treatment by administration of an effective dose of the at least one strain of Lactobacillus.

Fractalkine

Fractalkine is the only known member of the CX₃C chemokine family, characterised in having three amino acids separating the cysteines near the N-terminus. In mice, fractalkine is also known as neurotactin. Membrane-bound fractalkine (CX3CL1) has 373 amino acids (human mature length after removal of the signal peptide) comprising an N-terminal domain (residues 1-76), a mucin-like stalk (residues 77-317), a transmembrane alpha helix (residues 318-336) and a cytoplasmic tail (residues 337-373) (Jones et al, 2010, Mol Interv, 10(5):263-70; Bazan et al, 1997, Nature, 385(6617):640-644; the entire contents of both of which are incorporated herein by reference).

Major sites of fractalkine expression are neurons and epithelial cells in the lung, kidney and intestine, and fractalkine can also be expressed by endothelial and smooth muscle cells under inflammatory conditions (White et al, 2012, supra). Membrane-bound fractalkine expressed on activated endothelial cells promotes strong adhesion of leukocytes. Fractalkine elicits its adhesive and migratory functions by interacting with the chemokine receptor CX3CR1, e.g. on the surface of NK cells, cytotoxic T lymphocytes and gamma-delta cells. Membrane-anchored fractalkine is also expressed on monocytes during inflammatory conditions, suggesting that fractalkine plays a role in inflammatory diseases (Jones et al, 2010, supra). Membrane-anchored fractalkine expressed on neurons has been found to be essential for microglial cell migration, as microglial cells express the CX3CR1 receptor. Fractalkine is also up-regulated in the hippocampus during a brief temporal window following spatial learning.

A soluble 95 kDa form of fractalkine (soluble fractalkine or processed fractalkine) contains the chemokine domain and the extracellular mucin-like stalk. Soluble fractalkine is released via cleavage of membrane-bound fractalkine at the base of the mucin stalk by the shedding proteases ADAM10 or ADAM17 (Zunke et al, 2017, Biochim Biophys Acta Mol Cell Res, 1864(11 Pt B):2059-2070). Shedding by ADAM10 is believed to be constitutive, whereas shedding by ADAM17 is in response to cell activation, such as in inflammatory conditions (White et al, 2012, supra). At least two cleavage sites for ADAM10 are known (White et al, 2012, Arterioscler Thromb Vasc Biol, 32:589-594). Soluble fractalkine potently chemoattracts T cells, monocytes and natural killer (NK) cells.

The amount of fractalkine and its receptor CX3CR1 has been shown to be increased in several forms of cancer, inflammatory diseases and cardiovascular diseases. For example, increased levels of soluble fractalkine have been reported in patients with ruptured coronary plaques (Ikejima et al, 2010, Circ J, 74:337-345) and fractalkine is implicated in cardiovascular disease by a proliferative and anti-apoptotic effect on primary human smooth muscle cells (White et al, 2010, Cardiovasc Res, 85:825-835). Increased levels of soluble fractalkine have also been reported in synovial fluid in rheumatoid arthritis (Ruth et al, 2001, Arthritis Rheum, 44:1568-1581). Intestinal epithelial cells expressing membrane-anchored fractalkine have been shown to shed soluble fractalkine in response to stimulation by IL-1β (Muehlhoefer et al, 2000, J Immunol, 164:3368-3376). Further, human intestinal microvascular endothelial cells have been shown to shed soluble fractalkine in response to stimulation by a combination of IFN-γ and TNFα but not by IFN-γ or TNFα alone, which was higher in cells from patients with ulcerative colitis and Crohn's disease than from healthy controls (Sans et al, 2007, Gastroenterology, 132(1):139-53).

Administration of anti-fractalkine monoclonal antibody in two murine models of inflammatory bowel disease showed positive results including suppression of body weight loss, suggesting a role of fractalkine in the pathogenesis of IBD (Nishimura et al, 2009, Ann NY Acad Sci, 1173:350-6). Nishimura et al., 2009 highlights the biological significance of the fractalkine-fractalkine receptor (CX3CL1-CX3CR1) interaction in the development of colitis.

The colons of patients with inflammatory bowel disease show increased levels of fractalkine and high numbers of fractalkine receptor-positive cells. Injection of mice with an anti-fractalkine monoclonal antibody, to disrupt the fractalkine-fractalkine receptor pathway, efficiently inhibited intravascular monocyte patrolling behaviour; reduced the expression of inflammatory cytokines in the colon; maintained blood vessel integrity and significantly suppressed the colitis symptoms in oxa-induced mice colitis models (Kuboi et al. 2019, Int Immunol. 2019 Apr. 26; 31(5):287-302). The anti-fractalkine antibody also ameliorated symptoms in another IBD model, T-cell-transferred colitis. The results in Kuboi et al. 2019 indicate that the fractalkine-fractalkine receptor axis plays a critical role in the pathogenesis of murine colitis models and suggests that disruption of the fractalkine-fractalkine receptor interaction and associated pathways may be an effective strategy in treating inflammatory bowel diseases (IBDs), including Crohn's disease (CD) and ulecerative colitis (UC).

Fractalkine and its receptor play a role in the chemotherapy-induced peripheral neuropathy (CIPN). When fractalkine and the fractalkine-receptor are highly expressed, they activate spinal microglia, which then produce inflammation factors such as TNF-α and IL-1b via P38/MAPK signalling pathway, leading to CIPN (Clark and Malcangio, 2012, Exp Neurol. 2012 April; 234(2):283-92). Gastrodin relieved CIPN induced in mice by inhibiting the activation of spinal microglia via decreasing the fractalkine and fractalkine receptor pathway (Qin et al., Drug Chem Toxicol. 2018 Dec. 17:1-8).

The pathophysiologic mechanisms of diabetic retinopathy (DR) are complicated and closely related and are likely linked to oxidative stress, NF-κB and inflammatory mediators. The expression of a variety of oxidative stress and inflammatory mediators (including fractalkine) were assessed in diabetes-induced rats using RT-PCR, western blot analysis and enzyme-linked immunosorbent assays (ELISA). Some rats received treatment with cilostazol. In the retinas of diabetic rats, the levels of fractalkine mRNA in diabetic mice were elevated five-fold and fractalkine expression was elevated by four-fold in comparison to healthy rats. Treatment with cilostazol reduced the levels of fractalkine mRNA by 66% and fractalkine expression by 62%. Other markers of oxidative stress and inflammation were monitored and cilostazol was seen to reduce inflammatory reactions and oxidative stress in diabetic eyes. The anti-inflammatory effects of cilostazol may be indirectly via reducing oxidative stress, inhibiting NF-κB activity, and subsequently decreasing inflammatory mediators. Cilostazol may be beneficial to prevent the progression of diabetic retinopathy via these mechanisms (Yeh et al. Curr Eye Res 2019 March; 44(3):294-302).

Hence, it will be appreciated that reduction of soluble fractalkine may be advantageous in providing protection against and/or treatment of the conditions or diseases in which elevated soluble fractalkine is implicated. However, before the present disclosure, no link had been made between acute psychosocial stress and fractalkine release.

In a most preferred embodiment, administration of the at least one strain according to the invention may reduce or prevent an elevated level of soluble fractalkine in plasma as a deleterious effect of acute psychosocial stress in a human, and thereby provide protection against and/or relief from disorders, conditions or diseases in which elevated soluble fractalkine is implicated, such as cancer, chemotherapy-induced peripheral neuropathy (CIPN), diabetic retinopathy (DR), inflammatory disease, cardiovascular diseases, inflammatory bowel disease (IBD), irritable bowel syndrome (IBS), ulcerative colitis and Crohn's disease.

Patient Group

It will be appreciated that the at least one strain according to the invention may be useful in reducing and/or preventing at least one deleterious effect of acute psychosocial stress in any human that experiences acute psychosocial stress.

In particular, the at least one strain is suitable to be used in humans (men and/or women), including adolescents (aged 12-17 years old), young adults (aged 18-30 years old), and adults older than 30, 40, 50, 60, 70 and 80 years old.

The human to be treated may be an individual that has chronic stress or an individual that does not have chronic stress.

By “chronic stress” we include the meaning of a score of 3.75 or greater in the Shirom-Melamed Burnout Questionnaire (SMBQ) (Grossi et al, 2003, J Psychosomatic Res 55: 309-316). Hence, the human to be treated may have chronic stress indicated by a score of 3.75 or greater in the SMBQ. Alternatively, the human to be treated may not have chronic stress, indicated by a score of less than 3.75 in the SMBQ.

It will also be appreciated that “chronic stress” is not intended to include depression. By depression we include the meaning of a score of 45 or greater in the Zung Self-Rating Depression Scale (Zung, 1965, Arch Gen Psychiatry, 12:63-70, the entire contents of which are incorporated herein by reference

Lactobacillus Strains

The at least one Lactobacillus strain according to the invention may be viable, inactivated or dead. Preferably, the strain is viable. For example, preferably the strain is freeze-dried.

Advantageously, the at least one strain of Lactobacillus is a probiotic strain.

Probiotic bacteria are defined as “live microorganisms that, when administered in adequate amounts, confer a health benefit on the host” (Hill et al, Nat Rev Gastroenterol Hepatol, 2014, 11(8):506-514). For a bacterium to fulfil the definition of a probiotic it typically has to be able to survive in and colonise the intestines, survive the processes of production and storage, and have evidence that it has positive effects on consumer health.

By “at least one probiotic strain” we include the meaning of one or more strain(s) of bacteria which when administered in adequate amounts confer a health benefit on the host. Preferably the “at least one strain” according to the first aspect of the invention is selected from the species Lactobacillus plantarum, Lactobacillus paracasei, Lactobacillus rhamnosus, Lactobacillus crispatus, Lactobacillus gasseri, Lactobacillus fermentum, Lactobacillus reuteri, Lactobacillus acidophilus, Lactobacillus helveticus, Lactobacillus casei, Lactobacillus salivarius, and Lactobacillus johnsonii

Preferably the “at least one strain” according to the invention is at least one strain of Lactobacillus plantarum.

Preferably, the at least one strain of Lactobacillus plantarum is chosen from Lactobacillus plantarum DSM 15312 (HEAL9™) Lactobacillus plantarum DSM 15313 (HEAL 19™), Lactobacillus plantarum DSM 15316 (HEAL 99™) Lactobacillus plantarum DSM 6595 (299™), Lactobacillus plantarum DSM 9843 (299v®), Lactobacillus plantarum DSM 32131 (GOS42™), Lactobacillus plantarum DSM 17852 (LB3e™), and Lactobacillus plantarum DSM 17853 (LB7c™).

Lactobacillus plantarum DSM 15312 (HEAL 9™), Lactobacillus plantarum DSM 15313 (HEAL 19™), and Lactobacillus plantarum DSM 15316 (HEAL 99™) were deposited on 27 Nov. 2002 at DSMZ-DEUTSCHE SAMMLUNG VON MIKROORGANISMEN UND ZELLKULTUREN GmbH, Mascheroder Weg 1b, D-38124 Braunschweig, Germany, by Probi AB.

Lactobacillus plantarum DSM 6595 (299™) was deposited on 2 Jul. 1991 at DSM-DEUTSCHE SAMMLUNG VON MIKROORGANISMEN UND ZELLKULTUREN GmbH, Mascheroder Weg 1 B, D-3300 Braunschweig, Germany, in the name of Probi (i.e. Probi AB).

Lactobacillus plantarum DSM 9843 (299v®) was deposited on 16 Mar. 1995 at DSM-DEUTSCHE SAMMLUNG VON MIKROORGANISMEN UND ZELLKULTUREN GmbH, Mascheroder Weg 1 b, D-38124 Braunschweig, Germany, by Probi AB.

Lactobacillus plantarum DSM 32131 (GOS42™) was deposited on 2 Sep. 2015 at Leibniz Institute DSMZ-German Collection of Microorganisms and Cell Cultures, Inhoffenstr. 7 B, D-38124 Braunschweig, Germany by Probi AB.

Lactobacillus plantarum DSM 17852 (LB3e™) and Lactobacillus plantarum DSM 17853 (LB7c™) were deposited on 6 Jan. 2006 at DSMZ-Deutsche Sammlung von Mikroorganismen and Zellkulturen GmbH, Mascheroder Weg 1b, D-38124 Braunschweig, Germany, by Probac AB. All rights and duties in connection with microorganism deposits DSM 17852 and DSM 17853 were subsequently given to and accepted by Probi AB, who is now the depositor of the DSM 17852 and DSM 17853 strains.

Preferably, the at least one strain of Lactobacillus plantarum is able to adhere to the intestinal epithelium and persist in the intestine. Particular strains of this species comprise a mannose-specific adhesin (Adlerberth et al, 1996, Appl Environ Microbiol, 62(7):2244-2251, the entire contents of which are incorporated herein by reference), including Lactobacillus plantarum DSM 15312 (HEAL 9™), Lactobacillus plantarum DSM 15316 (HEAL 99™) Lactobacillus plantarum DSM 9843 (299v®) and Lactobacillus plantarum DSM 6595 (299™).

Most preferably, the at least one strain of Lactobacillus plantarum is Lactobacillus plantarum DSM 15312 (HEAL 9™).

Compositions

The at least one strain according to the invention may be present in a composition comprising at least one suitable carrier. For example, the carrier may be a diluent or excipient. The composition may be as a solid or liquid formulation, and hence the at least one carrier may be a solid or a liquid, or may comprise both at least one solid component and at least one liquid component.

Examples of a suitable liquid carrier include water, milk, coconut water, fruit drinks and juices, milk substitutes (soya drink, oat drink, nut and other plant-based drinks), sparkling beverages, oil formulations, including one or more of a nut or vegetable oil, such as coconut, rapeseed, olive, corn/maize; palm; glycerin, propylene glycol and other aqueous solvents.

Examples of a suitable solid carrier or excipient include maltodextrin, inulin, a cellulose such as microcrystalline cellulose (MCC), hydroxypropylmethylcellulose (HPMC) or hydroxy-propylcellulose (HPC), sugar alcohols, high molecular weight polyethylene glycols, lactose, sodium citrate, calcium carbonate, dibasic calcium phosphate and glycine, disintegrants such as starch (preferably corn, potato, tapioca or other vegetable starch), sodium starch glycollate, croscarmellose sodium and certain complex silicates, and granulation binders such as polyvinylpyrrolidone, sucrose, gelatin and acacia. Additionally, lubricating agents such as magnesium stearate, stearic acid, glyceryl behenate and talc may be included.

In an embodiment according to the first aspect of the invention, the carrier may be selected from a pharmaceutically and/or nutritionally (food-grade) acceptable carrier, or excipient. For example, the carrier material may be a food.

Examples of suitable pharmaceutically acceptable carriers, excipients and diluents include those well known to a skilled person in the art, for example those given in Remington: The Science and Practice of Pharmacy, 19^{th} ed., vol. 1 & 2 (ed. Gennaro, 1995, Mack Publishing Company).

By “food-grade” we include carriers, ingredients and excipients that meet the ‘generally recognized as safe’ (GRAS) criteria.

By “food” we include any substance for consumption to provide nutritional benefit or support for an organism. Examples of suitable food carriers include beverages (e.g. juices), dairy products (e.g. yoghurts, cheese, ice creams, infant formula and spreads such as margarine), dairy-alternative products (e.g. soy, nut or other plant-based drinks, yoghurts and spreads), cereal-based products (e.g. breads, biscuits, breakfast cereals, pasta and dry food bars such as health bars), and baby food (e.g. pureed fruit and/or vegetable).

The composition according to the invention may be a dry, non-fermented composition, a fermented composition, or a dry, fermented composition. Fermentation in this context particularly includes lactic acid fermentation by lactic acid bacteria in anaerobic conditions. In the case of a dry, non-fermented composition, substantially no fermentation takes place before ingestion by a subject, and so fermentation only takes place in the gastrointestinal tract after ingestion of the composition by a subject.

Hence, in some embodiments according to the invention, the composition is in the form of a food wherein the food is a cereal-based product, a dairy product, a juice drink, or a fermented food.

Examples of fermented foods include fermented milk products (such as yoghurt, kefir or lassi), fermented dairy-free milk alternatives (such as coconut milk kefir), fermented cereal-based products (such as oats, oatmeal, maize, sorghum, wheat), fermented vegetables (such as sauerkraut, kimchi, or pickles), fermented legumes or soybeans (such as natto or tempeh) and fermented tea (such as kombucha). Other examples of dairy free milk alternatives include almond, soy and oat-based “ghurts”.

In some embodiments according to the invention, the at least one strain is present in a composition that is not naturally occurring, e.g. the composition comprises more than the strain(s) and water.

In use, the at least one strain or the composition comprising the at least one strain according to the invention may be mixed with a liquid or solid carrier before administration to a mammal. For example, a subject may mix the at least one strain or the composition thereof with a carrier comprising one or more liquids chosen from water, milk, coconut water, fruit drinks and juices, milk substitutes (soya drink, oat drink, nut and other plant-based drinks), sparkling beverages or some other aqueous solvent or drink prior to intake. Similarly, the at least one strain or the composition thereof may be mixed with a carrier consisting of one or more foods. Suitable food carriers include oatmeal carrier, barley carrier, fermented or non-fermented dairy products such as yoghurts, ice creams, milkshakes, fruit juices, beverages, soups, breads, biscuits, pasta, breakfast cereals, dry food bars including health bars, plant-based foods such as soy products, spreads, baby food, infant nutrition, infant formula, breast milk replacements from birth.

Preferably, the formulation is a unit dosage containing a daily dose or unit, daily sub-dose or an appropriate fraction thereof, of the composition comprising the strain.

The composition according to the invention may be a dietary supplement. By “dietary supplement” we include the meaning of a manufactured product intended to supplement the diet when taken by mouth, e.g. as a pill, capsule, tablet, or liquid. Dietary supplements may contain substances that are essential to life and/or those that have not been confirmed as being essential to life but may have a beneficial biological effect. When the composition according to the invention is in the form of a dietary supplement the carrier(s) to be added include those well known to a skilled person in the art, for example those given in Remington: The Science and Practice of Pharmacy, 19^{th} ed., vol. 1 & 2 (ed. Gennaro, 1995, Mack Publishing Company). Any other ingredients that are normally used in dietary supplements are known to a skilled person and may also be added conventionally together with the at least one strain.

The composition according to the invention may be provided in the form of a solution, suspension, emulsion, tablet, granule, powder, capsule, lozenge, chewing gum, or suppository.

For example, in a preferred embodiment, the composition according to the invention is a dietary supplement in the form of a capsule comprising freeze-dried Lactobacillus.

In an embodiment according to the invention, the at least one strain is present (e.g. in a composition) in an amount from about 1×10⁸ to about 1×10¹⁴ CFU/dose, preferably from about 1×10⁸ to about 1×10¹² CFU/dose, more preferably from about 1×10⁹ to about 1×10¹¹ CFU/dose, and most preferably about 1×10¹⁰ CFU/dose. If the at least one strain consists of more than one strain, such amounts represent the total CFU/dose of the combination of strains. For example, the at least one strain may be present in an amount from about 1×10⁶, 1×10⁷, 1×10⁸, 1×10⁹, 1×10¹⁰, 1×10¹¹, 1×10¹² or about 1×10¹³ CFU/dose. The at least one strain may be present in an amount to about 1×10¹⁴, 1×10¹³, 1×10¹², 1×10¹¹, 1×10¹⁰, 1×10⁹, 1×10⁸ or about 1×10⁷ CFU/dose. The at least one strain according to the invention may also be used alone in water or any other aqueous vehicle in which the at least one strain is added or mixed before ingestion.

The composition according to the invention can be administered orally, buccally or sublingually in the form of tablets, capsules, powders, ovules, elixirs, solutions or suspensions, which may contain flavouring or colouring agents, for immediate-, delayed- or controlled-release applications. The composition may be administered in the form of a powdered composition such as a fast-melt microbial composition, for example those described in WO 2017/060477, or in Probi's UK Patent Application 1708932.7 or Probi's publication WO 2018/224509 relating to Probi® Fast Melt technology, the entire contents of all three of which are incorporated herein by reference. Where the powder is not in a fast-melt microbial composition, it may be suitable for being added to a food (e.g. yoghurt) or drink (e.g. water or milk) before ingestion.

Where the composition is in the form of a powder, it would typically be filled in a sealed container, which provides an oxygen and moisture barrier in order to protect and maintain the viability of the bacteria in the composition. Hence, where the composition is in the form of a powder, preferably the composition is packaged in sealed aluminium foil sticks, where each stick comprises one dose of the composition, i.e. one dose of the bacteria. Non-limiting examples of suitable containers include a stick, bag, pouch or capsule. In a preferred embodiment, the container is an aluminium foil or a polyethylene stick, which is typically sealed by welding. The stick is typically configured for easy tear opening. The stick may have a tear notch.

The composition according to the invention may be formulated as a controlled-release solid dosage form, for example any of those described in WO 03/026687 and U.S. Pat. Nos. 8,007,777 and 8,540,980, the entire contents of which are incorporated herein by reference. The composition may be formulated as a layered dosage form, for example Probi's BIO-tract® technology including any of the layered dosage forms described in WO 2016/003870, the entire contents of which are incorporated herein by reference.

A tablet may be made by compression or moulding, optionally with one or more accessory ingredients. Compressed tablets may be prepared by compressing in a suitable machine the at least one probiotic strain (e.g. freeze-dried) in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

Methods of Treatment

The invention also provides a method for reducing and/or preventing at least one deleterious effect of acute psychosocial stress in a human, wherein the method comprises treating by administering to a human an effective dose of the at least one strain of Lactobacillus, or the composition thereof.

In particular, the methods of treatment include the methods described above for use in which is the at least one strain according to the aspects of the invention described above.

By “treat”, “treating” and “treatment” we include the meaning that the disease or condition being treated is ameliorated, reduced in severity, removed, blocked from occurring further, protected against occurring further, delayed and/or made to cease. The terms “treat”, “treating” and “treatment” also include the meaning of delaying, protecting against, reducing the severity of and/or removing, one or more symptoms, effects, indications and/or other markers associated with a given disease or condition. Where one of these terms is used in the context of a disease or condition, such treatment typically takes place after the disease or condition is manifest. It will also be appreciated that such terms may include the meaning that a disease or condition is maintained in the current state without becoming worse or developing further.

The human being treated by the methods according to the invention may be any human given above in relation to the earlier aspect of the invention. For example, the human may be a man or a woman, and may be an adolescent (aged 12-17 years old), a young adult (aged 18-30 years old), or an adult older than 30, 40, 50, 60, 70 or 80 years old.

Administration according to the methods of the invention may include administration orally, buccally or sublingually as described above in relation to the earlier aspect of the invention.

Administration according to the methods of the invention may include administration at least every one, two, three, four, five, six or seven days, or at least one, two, three, four, five, six or seven times a week. Preferably administration takes place at least once daily.

Administration according to the methods of the invention may include administration that is repeated for at least one, two, three, four, five or six days, or for at least one, two, three or four weeks. Preferably, administration is repeated at least once daily for at least 7 days, such as for at least one week, preferably for at least two weeks, more preferably for at least three weeks, and most preferably for at least four weeks.

Administration according to the methods of the invention is preferably of a unit dosage of from about 1×10⁶ to about 1×10¹⁴ CFU/unit dose, preferably from about 1×10⁸ to about 1×10¹² CFU/unit dose, more preferably from about 1×10⁹ to about 1×10¹¹ CFU/unit dose, and most preferably about 1×10¹⁰ CFU/unit dose, in accordance with the invention. Administration according to the methods of the invention preferably results in an effective dose of from about 1×10⁶ to about 1×10¹⁴ CFU/unit dose, preferably from about 1×10⁸ to about 1×10¹² CFU/unit dose, more preferably from about 1×10⁹ to about 1×10¹¹ CFU/unit dose, and most preferably about 1×10¹⁰ CFU/unit dose. Preferably, each subject is administered at least one unit dose per day, such as one unit dose per day. Hence, administration according to the methods of the invention preferably results in a daily dose of from about 1×10⁶ to about 1×10¹⁴ CFU/day, preferably from about 1×10⁸ to about 1×10¹² CFU/day, more preferably from about 1×10⁹ to about 1×10¹¹ CFU/day, and most preferably about 1×10¹⁰ CFU/day.

It will be appreciated that a preferable daily dose may also be achieved by administration of more than one sub-dose, for example, by a twice daily administration of a unit dose comprising half of the preferable daily dose. Hence, the preferred ranges for the effective dose may also represent the preferred daily dosage to be achieved in whatever number of unit doses is practical.

The subject may be instructed to consume the therapeutically effective amount of the at least one strain according the invention or the composition according to the first aspect of the invention, in combination with water, another aqueous solvent or a food product, e.g. yoghurt.

It will be appreciated that the methods according to the present invention should preferably be carried out before the episode(s) of acute psychosocial stress so that the at least one strain according to the present invention can have effect to reduce and/or prevent at least one deleterious effect of acute psychosocial stress caused by the subsequent acute psychosocial stressor. Hence, preferably administration of the at least one strain according to the invention begins at least 1, 2, 3, 4, 5 or 6 days before the episode of acute psychosocial stress, more preferably at least 1, 2, 3 or 4 weeks before the episode of acute psychosocial stress.

In a preferred embodiment according to the invention, the method for reducing and/or preventing at least one deleterious effect of acute psychosocial stress in a human comprises treating by administering to a human 10¹⁰ CFU of the at least one strain according to the invention or a composition thereof, for at least 30 days.

By reducing and/or preventing at least one deleterious effect of acute psychosocial stress in a human, the methods of treatment according to the invention may be effective to treat, prevent or reduce at least one symptom of a disease, disorder, or condition. In particular, by reducing and/or preventing an elevated level of soluble fractalkine in plasma as a deleterious effect of acute psychosocial stress in a human, the methods of treatment according to the invention may be effective to treat, prevent or reduce at least one symptom of one or more diseases, disorders, or conditions selected from cancer, chemotherapy-induced peripheral neuropathy (CIPN), diabetic retinopathy (DR), inflammatory disease, cardiovascular diseases, inflammatory bowel disease (IBD), irritable bowel syndrome (IBS), ulcerative colitis and Crohn's disease. In such methods according to the invention, preferably the deleterious effect of acute psychosocial stress is an elevated level of soluble fractalkine, particularly in plasma.

Use in a Method

A further aspect of the invention provides the use of a composition comprising the at least one strain according to the invention or a composition thereof, in a method of reducing and/or preventing at least one deleterious effect of acute psychosocial stress in a human. In particular, the method is a method according to the above methods of the invention.

The listing or discussion of an apparently prior-published document in this specification should not necessarily be taken as an acknowledgement that the document is part of the state of the art or is common general knowledge.

The invention will now be described in more detail by reference to the following Examples and Figures.

### BRIEF DESCRIPTION OF THE FIGURES

FIG. 1 shows the change in cortisol levels In(nmol/L) after the TSST test. LPHEAL9 as solid line; PLACEBO as dotted line. The dashed line at zero represents the level in the first sample before the TSST challenge (BASE), and each time point represents the change from that BASE reading: PREP=second sample before the TSST challenge; v-TSST=directly after the TSST challenge; R+10, R+20, R+30, R+40, R+60=10, 20, 30, 40 and 60 minutes after the TSST challenge within the recovery phase.

FIG. 2 shows the change in fractalkine levels sqrt(pg/mL) after the TSST test. The lines and x-axis conditions are the same as FIG. 1.

FIG. 3 shows the change in soluble CD163 levels (log 10[ng/mL]) after the TSST test. The lines and x-axis conditions are the same as FIG. 1, except that results are not shown for R+60.

FIG. 4 shows the change in fractalkine levels (pg/mL) after the TSST test, with a two-factor comparison of (1) subjects with an SMBQ value of 3.75 (chronic stress, HS) on the test day vs subjects with an SMBQ value of <3.75 (low stressed, LS) on the test day, and (2) subjects in the “Active” (L. plantarum DSM 15312 pre-treated) group vs subjects in the “Placebo” control group. Zero on the y-axis represents the level in the first sample before the TSST challenge (BASE), and each time point represents the change from that BASE reading: 0=v-TSST (directly after the TSST challenge); 10, 20, 30, 40, 60=10, 20, 30, 40 and 60 minutes after the TSST challenge within the recovery phase.

FIG. 5 shows the change in soluble CD163 levels (ng/mL) after the TSST test. The comparison and graphical representation details are the same as for FIG. 4.

FIG. 6 shows the change in cortisol levels (nmol/L) after the TSST test. The comparison and graphical representation details are the same as for FIG. 4.

### EXEMPLARY DOSAGE FORMS

In addition to the formulations referenced above (and incorporated herein by reference), the following examples illustrate pharmaceutical formulations and other formulations according to the invention.

### Example A: Tablet

| | |
|---|---|
| Lactobacillus strain(s) | 1 × 10¹⁰ CFU |
| Lactose | 200 mg |
| Starch | 50 mg |
| Polyvinylpyrrolidone | 5 mg |
| Magnesium stearate | 4 mg |

Tablets are prepared from the foregoing ingredients by wet granulation followed by compression.

### Example B: Tablet Formulations

The following formulations A and B are prepared by wet granulation of the ingredients with a solution of povidone, followed by addition of magnesium stearate and compression.

Formulation A

| | | |
|---|---|---|
| (a) Lactobacillus strain(s) | 1 × 10¹⁰ CFU | 1 × 10¹⁰ CFU |
| (b) Lactose B.P. | 210 mg | 26 mg |
| (c) Povidone B.P. | 15 mg | 9 mg |
| (d) Sodium Starch Glycolate | 20 mg | 12 mg |
| (e) Magnesium Stearate | 5 mg | 3 mg |

Formulation B

| | | |
|---|---|---|
| (a) Lactobacillus strain(s) | 1 × 10¹⁰ CFU | 1 × 10¹⁰ CFU |
| (b) Lactose | 150 mg | — |
| (c) Avicel PH 101 ^{®} | 60 mg | 26 mg |
| (d) Povidone B.P. | 15 mg | 9 mg |
| (e) Sodium Starch Glycolate | 20 mg | 12 mg |
| (f) Magnesium Stearate | 5 mg | 3 mg |

Formulation C

| | |
|---|---|
| Lactobacillus strain(s) | 1 × 10¹⁰ CFU |
| Lactose | 200 mg |
| Starch | 50 mg |
| Povidone | 5 mg |
| Magnesium stearate | 4 mg |

The following formulations, D and E, are prepared by direct compression of the admixed ingredients. The lactose used in formulation E is of the direction compression type.

Formulation D

| | |
|---|---|
| Lactobacillus strain(s) | 1 × 10¹⁰ CFU |
| Pregelatinised Starch NF15 | 150 mg |

Formulation E

| | |
|---|---|
| Lactobacillus strain(s) | 1 × 10¹⁰ CFU |
| Lactose | 150 mg |
| Avicel ^{®} | 100 mg |

Formulation F (Controlled Release Formulation)

The formulation is prepared by wet granulation of the ingredients (below) with a solution of povidone followed by the addition of magnesium stearate and compression.

| | |
|---|---|
| (a) Lactobacillus strain(s) | 1 × 10¹⁰ CFU |
| (b) Hydroxypropylmethylcellulose | 112 mg |
| (Methocel K4M Premium) ^{®} | |
| (c) Lactose B.P. | 53 mg |
| (d) Povidone B.P.C. | 28 mg |
| (e) Magnesium Stearate | 7 mg |

Release takes place over a period of about 6-8 hours and was complete after 12 hours.

### Example C: Capsule Formulations

Formulation A

A capsule formulation is prepared by admixing the ingredients of Formulation D in Example B above and filling into a two-part hard gelatin capsule. Formulation B (infra) is prepared in a similar manner.

Formulation B

| | |
|---|---|
| (a) Lactobacillus strain(s) | 1 × 10¹⁰ CFU |
| (b) Lactose B.P. | 143 mg |
| (c) Sodium Starch Glycolate | 25 mg |
| (d) Magnesium Stearate | 2 mg |

Formulation C

| | |
|---|---|
| (a) Lactobacillus strain(s) | 1 × 10¹⁰ CFU |
| (b) Macrogol 4000 BP | 350 mg |

Capsules are prepared by melting the Macrogol 4000 BP, dispersing the Lactobacillus strain(s) in the melt and filling the melt into a two-part hard gelatin capsule.

Formulation D (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | |
|---|---|
| (a) Lactobacillus strain(s) | 1 × 10¹⁰ CFU |
| (b) Microcrystalline Cellulose | 125 mg |
| (c) Lactose BP | 125 mg |
| (d) Ethyl Cellulose | 13 mg |

### Example D: Powder formulations

Formulation A (fast-melting microbial composition)

| | |
|---|---|
| (a) Lactobacillus strain(s) | 80 mg (preferably 1 × 10¹⁰ CFU) |
| (b) Erythritol | 450 mg |
| (c) Inulin | 227.5 mg |
| (d) Xylitol | 227.5 mg |
| (e) Lemon flavour | 10 mg |
| (f) Silicon dioxide | 5 mg |

Formulation B (fast-melting microbial composition)

| | |
|---|---|
| (a) Lactobacillus strain(s) | 80 mg (preferably 1 × 10¹⁰ CFU) |
| (b) Erythritol | 425 mg |
| (c) Inulin | 215 mg |
| (d) Xylitol | 215 mg |
| (e) Maltodextrin | 50 mg |
| (f) Lemon flavour | 10 mg |
| (g) Silicon dioxide | 5 mg |

### Experimental Example 1

Introduction

Stress for a long time (chronic stress), and stress-related health problems are a source of concern in many postindustrial countries as it affects e.g. the individual well-being, the healthcare system and contributes to sickness absence. To gain further knowledge about stress-related health problems, the Trier Social Stress Test (TSST) is a widely used tool to induce psychosocial stress in a laboratory setting (Kirschbaum et al, 1993, Neuropsychobiology, 28(1-2):76-81).

The purpose of this study was to investigate how a Lactobacillus strain affects stress markers, bowel health and inflammation when high stressed persons (suffering from chronic stress) take the study product four weeks before being exposed to an acute stressful situation, TSST in the virtual reality laboratory at the Department of Design Sciences (Ingvar Kamprad Design Center, Faculty of Engineering [LTH], Lund University, Sweden).

Objectives

Primary Objective
To assess whether intake of Lactobacillus plantarum DSM 15312 (HEAL 9™) can counteract elevated levels of serum cortisol in subjects with chronic stress that are exposed to acute stress (TSST).

Secondary Objectives
To assess whether intake of Lactobacillus plantarum DSM 15312 (HEAL 9™) can reduce the increase in interleukin-6 and other inflammation markers in subjects with chronic stress that are exposed to acute stress (TSST);
To assess whether intake of Lactobacillus plantarum DSM 15312 (HEAL 9™) affects the permeability of the gut (zonulin) in subjects with chronic stress that are exposed to acute stress (TSST);
To assess whether intake of Lactobacillus plantarum DSM 15312 (HEAL 9™) can influence physiological data like pulse and heart rate variability in subjects with chronic stress that are exposed to acute stress (TSST);
To evaluate changes over time (4 weeks and before/after the TSST, respectively) in abdominal pain, flatulence and bloating;
The frequency of adverse events (AEs) collected during the study.

Investigational Plan

Overall Study Design

The study was a prospective, double-blind, placebo-controlled, parallel, single-centre study conducted in healthy adults (19-35 years) with and without chronic stress. The duration of the study was 6 weeks, consisting of two phases:
Phase 1: Run-in for two weeks. No intake of probiotic products.
Phase 2: Intake of Lactobacillus plantarum DSM 15312 (HEAL 9™) OR placebo for four weeks. TSST challenge performed at Day 30±3.

Hence, after a two-week run-in period, the subjects were randomized to either consume a placebo capsule or a capsule with Lactobacillus plantarum DSM 15312 (HEAL 9™) once daily for 30 days. A TSST challenge was performed at Day 30±3.

Selection of Study Population

70 men and women with high levels of perceived stress were recruited.

In order for the results to be as comparable as possible, the recruited women made, if possible, the TSST in the luteal phase of their menstrual cycle. Women were asked if they used contraception.

Inclusion Criteria

The subjects met the following criteria:
1. 19-35 years;
2. Shirom-Melamed Burnout Questionnaire (SMBQ) score 3.75;
3. Understand Swedish in spoken and written terms;
4. Written informed consent in connection with study inclusion and obtaining serial number and study product (active or placebo);
5. No intake of probiotic product two weeks before start of study product introduction or during the entire ongoing study.

Exclusion Criteria

The subjects did not meet any of the following criteria:
1. BMI>30;
2. Pregnancy;
3. Antibiotics consumed during the study or three months prior to the start of study product introduction;
4. Previous or ongoing contact with health care due to stress-related problems;
5. Known physical (diabetes, pulmonary or cardiovascular disease, celiac disease, thyroid problems, gastrointestinal disease) or mental illness;
6. Consumption of psychotropics, beta blockers, asthma or rheumatoid drugs, steroid drugs or creams containing cortisone.

Exclusion Criteria During the Study was
1. Treatment with antibiotics;
2. Subject could not come on the test day and a new day could not be booked;
3. Adverse event—subject did not want to continue.

Dietary Regulations

During their participation in the study all subjects were instructed to follow certain dietary regulations that involved not consuming other products containing probiotics. A list containing the products not to be consumed was given to the study participants.

Treatments

Administration of Treatment

All capsules needed for one phase were handed out at Visit 1. Leftover capsules were collected and counted. The study product was packed externally and special personnel at

Probi AB that were not involved in the study were responsible for the labelling.

Identity of Study Products

The study product consisted of capsules containing either freeze dried Lactobacillus plantarum DSM 15312 (HEAL 9™) at a concentration of 10¹⁰ cfu/capsule or placebo capsules without the bacteria. The filler used in the capsules was maize starch and magnesium stearate. Both the probiotic and the placebo product were a white powder and had a similar appearance, texture and taste. The probiotic mixture contained traces of soy. The study product was taken once daily in connection with the breakfast by putting the capsule in the mouth and chewing it.

Methods of Assigning Subjects to Treatment Groups

The study product was prepared in labelled packages of 40 capsules per subject and labelling was done according to the corresponding randomization list (701, 702, . . . ). Study personnel provided the subjects with study product in the order they were randomised.

Blinding

The study was double-blind and the subject, study personnel and Probi personnel involved in the study did not know which product was distributed to each subject. Breaking the code was allowed in emergency situations but no blind was broken during the study.

Prior and Concomitant Medication

Medication was allowed during the study and was recorded in the study diary.

Treatment Compliance

At the end of the study participants were asked to hand in the remaining capsules that were counted to verify that the right amount of capsules had been ingested. The participants also had to note intake of study product in the study diary. Per protocol analysis was conducted on subjects who had taken more than 80% of the study product.

Efficacy and Safety variables

Study Flow Chart

The activities performed at each visit are shown in the study flow-chart (Table 1).

TABLE 1
Schedule of investigators' assessments

| Visit number | Visit 1 | Visit 2 |
|---|---|---|
| Day | −14 | 30 ± 3 |
| Informed consent | X | |
| Inclusion and exclusion criteria | X | |
| Medical history | X | |
| Physical examination | X | |
| (weight, height) | | |
| Questionnaire gut function | X | X |
| Sampling of saliva for analysis | X | X |
| of lactobacilli | | |
| Randomisation | X | |
| Diary distribution | X | |
| Study product distribution | X | |
| Self-Assessment Questionnaire | | X |
| Study product collection | | X |
| TSST challenge | | X |
| Medical interview (diseases, | | X |
| medication, food supplements, | | |
| probiotics) | | |
| Subject diary check | | X |
| Adverse Events | | X |

Efficacy

Primary Endpoint

The primary endpoint was to assess whether intake of Lactobacillus plantarum DSM 15312 (HEAL 9™) can counteract elevated levels of serum cortisol in subjects with chronic stress that are exposed to acute stress (TSST). Cortisol was measured eight times at Visit 2; twice before the TSST challenge (BASE, PREP), directly after the challenge (R+0) and five times during the recovery phase (R+10, R+20, R+30, R+40, R+60).

Secondary Endpoints

Shirom-Melamed Burnout Questionnaire (SMBQ)

SMBQ was measured in the recruiting phase and before the TSST challenge at the end of the intervention (Visit 2, Day 28).

Heart Rate (HR) and High Frequency Heart Rate Variability (HF-HRV)

Heart rate (HR) and high frequency heart rate variability (HF-HRV) were measured during the TSST challenge at Visit 2. Measurements were made for 5 min in each condition: BASE, PREP, SPEECH, MATH, and during the four subsequent recovery periods (R+10, . . . R+40), i.e. 8 conditions.

Gut Permeability (Zonulin) Serum zonulin was measured three times at Visit 2; once before the TSST challenge (BASE), and two times during the recovery phase (R+10, R+60).

Inflammation Markers

Different inflammation markers like CRP and IL-6 were measured in plasma eight times at Visit 2; twice before the TSST challenge (BASE, PREP), directly after the challenge (R+0) and five times during the recovery phase (R+10, R+20, R+30, R+40, R+60).

Gut Function

At Visit 1, basic questions about the gut function were asked. Further assessment of the gut function by filling in a visual analogue (VAS) scale (0-10) for abdominal pain, flatulence and bloating were done three times; once at Visit 1 and twice at Visit 2 (before and after the TSST challenge).

The Trier Social Stress Test (TSST)

The Trier Social Stress Test (TSST) is a widely used protocol for inducing social stress in laboratory settings (Kirschbaum et al, 1993, supra). Briefly, the test participant is asked to hold a speech and perform an arithmetic task in front of a committee. The committee consists of three actors who show no emotional responses to the test participant, making the situation very stressful. In the present study, TSST was performed in a virtual reality environment (V-TSST) according to Jönsson et al (2010, Psychoneuroendocrinology, 35(9):1397-1403), and conducted in the afternoon (1 μm to 5 μm) to avoid diurnal fluctuations of cortisol. The VR-TSST was created with a CAVE™ system (Cave Automatic Virtual Environment, Electronic Visualization Laboratory at the University of Illinois) with three rear-projected walls (4 m×3 m), and one floor projection. For 3D-vision, passive stereoscopy was used. Two virtual rooms were created using the software Autodeski 3 ds Maxi 9 and EON professional 5.5 (EON Reality, Inc.): a waiting room including a table, pictures on the walls, two chairs, a small table and a door on the opposite wall. Behind the door there was a room with one woman and two men (designed by the company aXYZ design) constituting the committee (Jönsson et al, 2010, supra; Wallerg∪rd et al, 2011, Presence, 20(4):325-336). Comments and instructions from the committee are given by prerecorded voices, in accordance with the standard TSST protocol (Kirschbaum et al, 1993, supra). Comments are activated by the test leader with a remote keyboard invisible to the test participant. For example, if the participant has difficulties continuing the presentation, the middle-aged man tells him that “you have time left,” or “please continue, I will tell you when your time is up.” The V-TSST has been shown to evoke reliable cortisol and cardiovascular responses in healthy women and men (Fich et al, 2014, Physiol Behav, 135:91-97; Jönsson et al, 2010, supra; Jönsson et al, 2015, Physiol Behav, 151:327-337).

Shirom-Melamed Burnout Questionnaire (SMBQ)

The SMBQ consists of 22 items that estimate four dimensions of burnout syndrome: burnout, tension, listlessness, and cognitive weariness (Melamed et al, 1992, Behav Med, 18(2):53-60). The SMBQ global score is represented by the mean of the four dimensions. The Swedish translation has previously been validated by Grossi et al (2003, J Psychosom Res, 55(4):309-16) and Lundgren-Nilsson et al (2012, BMC Public Health, 12:1). SMBQ was measured at inclusion and at the day when the VSST experiment took place.

Spielberger State and Trait Anxiety Inventory (STAI)

The state scale of STAI (STAI-S) was assessed before BASE and after 60 min of recovery to estimate the participants' experiences of V-TSST. At the second assessment, the instructions were slightly changed, from “answer the questions how you feel right now” to “answer to the

questions based on how you felt during the V-TSST”.

Heart Rate (HR)

Electrocardiography (ECG) and respiration were recorded at 1 kHz using the ML866 Power Lab data acquisition system and analyzed using its software Chart 5 (ADInstruments Pty Ltd.) and MATLAB (MathWorks, Inc., Natick, Mass.). ECG was assessed using disposable electrodes (Lead II Einthoven) and respiration using a strain gauge over the chest. Mean Heart Rate (HR) was analyzed for 5 min in each condition: BASE, PREP, SPEECH, MATH, and during the four subsequent resting periods (R+10, . . . R+40), i.e. 8 conditions (Jönsson et al, 2010, supra). The same applies to HF-HRV below.

High Frequency Heart Rate Variability (HF-HRV)

R-R intervals were transformed to a tachogram (ms) and linearly interpolated at 4 Hz. The data were further linearly detrended and highpass filtered (second order Butterworth filter, 0.10 Hz) to eliminate fluctuations below the respiratory frequency. For each 5-min sequence, HRV power spectra were calculated, for 17 segments of 128 points (32 s) with 50% overlap, using a fast Fourier transform (1024 points) following the application of multiple peak matched windows. The peak matched multiple windows (PM MW) method optimizes the mean square error of the spectrum estimate when the spectrum can be expected to include peaks (Hansson, 1999, IEEE Trans Signal Processing 47:1141-1146); Hansson and Salomonsson, 1997, IEEE Trans Signal Processing 45: 778-781)). The PM MW method has been shown to give reliable results for the HRV spectrum (Hansson-Sandsten and Jönsson, 2007, Transactions Biomed Engineering, 54: 1770-1779); Hansson and Jönsson, 2006, Medical Engineering & Physics, 28: 749-761). The integral of the power spectrum was studied in the high frequency (HF) region (0.12-0.4 Hz) that is related to respiration (Berntson et al., 1997, Psychophysiology, 34:623-648). The data were logtransformed (In) to approach a normal distribution. The respiration measures were used to ensure that the respiratory rate was within the HF range.

Biochemical Analyses

In order to verify intake of active study product versus placebo, oral lactobacilli were analysed. Saliva from the test subjects was collected at Visit 1 and Visit 2. Saliva and 2× Hogness Freezing Media (9.8 mM K₂HPO₄, 2.9 mM KH₂PO₄, 10.2 mM C₆H₅Na₃O₇ 2 (H₂O), 2.0 mM MgSO₄.7 (H₂O), 24.2% glycerol) was mixed in equal amounts before frozen and stored at −80° C.

Peripheral venous blood was collected eight times; twice before the TSST challenge (BASE, PREP), directly after the challenge (TSST; SPEECH+MATH) and five times during the recovery phase (REST+10 min, R+20, R+30, R+40 and R+60). Serum and plasma (plasma for analysis of cortisol and CRP) were separated at room temperature at 2000×g for 10 min. Plasma for analysis of cytokines was kept in ice bath and separated at 4° C. at 2000×g for 10 min (Eppendorf Centrifuge 5702R) within 30 min after sampling, using EDTA as anticoagulant.

Saliva, serum and plasma samples were frozen on dry ice and then stored at −80° C. until further analyses. Cortisol concentrations in serum were determined with a one-step competition assay with the Electrochemiluminiscence Immunoassay (ECLI) detection technique based on ruthenium derivate, and were conducted by Labmedicin Sk∪ne (university and regional laboratories in Region Sk∪ne, Sweden). Zonulin was analyzed in serum using an ELISA method (K5601, Immundiagnostik AG, Bensheim, Germany), detection range 0.25-64 ng/ml. The cytokines fractalkine, IFN-γ, IL-10, IL-1β, IL-6, IL-8, TNF-α, were analyzed in plasma using multiplex technology (U-PLEX human 7-plex [Fractalkine, IFN-γ, IL-10, IL-1β, IL-6, IL-8, TNF-α], MesoScale Discovery, Rockville Md., US) with detection limits 102 pg/mL, 1.7 pg/mL, 0.14 pg/μl, 0.15 pg/mL, 0.33 pg/mL, 0.15 pg/mL and 0.51 pg/mL, respectively. Soluble CD163 in plasma was analysed with an ELISA method (DY1607, R&D Systems, Minneapolis, US).

Safety

Reporting of Adverse Events

An Adverse Event (AE) is any untoward medical occurrence in a patient or clinical investigation subject administered a pharmaceutical product, which does not necessarily have a causal relationship with this treatment. An AE can therefore be any unfavourable and unintended sign (including an abnormal laboratory finding), symptom or disease temporarily associated with the use of a medicinal (investigational) product.

A Serious Adverse Event (SAE) is any untoward medical occurrence that at any dose:
results in death;
is life-threatening (the term ‘life-threatening’ in the definition of ‘serious’ refers to an event in which the patient is at risk of death at the time of the event; it does not refer to an event which hypothetically might have caused death if it were more severe);
requires inpatient hospitalisation or prolongation of existing hospitalisation;
results in persistent or significant disability/incapacity;
is a congenital anomaly/birth defect;
is an important medical event that requires intervention to prevent one of the above.

Adverse Events Causality

Investigator should try to assess the causality of an AE in relation to the study product as a whole. The following categories were used:
Probable: Apparent relationship in time between AE and study product administration. Relationship between AE and study product is already known or expected. Disappearance or diminution of symptoms after stopping the study product or reducing its dosage. The adverse reaction cannot be explained by the patient's clinical condition.
Possible: Apparent relationship in time between AE and drug administration. Relationship between AE and drug is already known or expected. Adverse reaction may also be explained by a number of other factors.
Unlikely: Relationship in time between AE and study product administration not probable. Adverse reaction to be explained rather by other factors, a relationship to the study product however cannot definitely be ruled out.

Detection, Reporting and Responsibilities

The obligation to report AEs started with enrolment of the subject in the study. Investigators were obliged to record all AE in subjects' clinical record describing date of onset, date stopped,

Data Sets to be Analysed

All correctly included and randomized subjects that underwent the TSST challenge were included in the full analysis set (FAS) and all randomized subjects that consumed at least one capsule of the study product were included in the safety set.

In order for the subjects to be included in the per-protocol set (PPS) they had to fulfil the following criteria:
The TSST challenge was carried out Day 30±3 in phase two;
At least 80% of the study product had been consumed;
No antibiotics were consumed during the study;
No other probiotic products had been consumed after the start of the study (minor amounts allowed).

Summary Statistics

In general, data have been summarized by means of summary statistics. Number of observations, mean value, standard deviation, median value, quartiles, minimum and maximum value have been presented (in Statistical Analysis Report, M Åstrom, 2018).

Summary statistics are presented by group.

Efficacy Analysis

Primary Efficacy Analysis

The primary endpoint was to assess whether intake of Lactobacillus plantarum DSM 15312 (HEAL 9™) can counteract elevated levels of serum cortisol in subjects with chronic stress that are exposed to acute stress (TSST).

The null hypothesis was that there are no differences in cortisol levels between the group that consumed Lactobacillus plantarum DSM 15312 (HEAL 9™) and the group that consumed placebo.

The alternative hypothesis is that there are differences in cortisol levels between the group that consumed Lactobacillus plantarum DSM 15312 (HEAL 9™) and the group that consumed placebo.

The analysis was done based on repeated measures ANOVAs with CONDITION as repeated factor and intervention (HEAL 9 and PLACEBO) as between group factor. Thus, an analysis of changes over time (CONDITION) was also included. AGE and BMI were used as covariates and entered stepwise. Any covariates that did not contribute significantly to each model were removed and were not reported. To meet the assumption of sphericity Greenhouse-Geisser correction was carried out and reported with corrected p-values, non-corrected df and ε. Significant omnibus effects were followed up with polynomial contrasts.

A comparison between HEAL 9 and PLACEBO using single time-points was also done as an exploratory analysis.

Secondary Efficacy Analysis

The secondary objectives included to assess whether intake of Lactobacillus plantarum DSM 15312 (HEAL 9™) can reduce the increase in zonulin, inflammation markers, pulse and heart rate in subjects with chronic stress that are exposed to acute stress (TSST). The same statistical methods as have been used for the primary variable were used.

Another secondary objective was to evaluate changes over time in abdominal pain, flatulence and bloating after consumption of Lactobacillus plantarum DSM 15312 (HEAL 9™) or placebo. The change in gut function before and after the TSST challenge was analysed using Wilcoxon rank sum test (comparison of intervention groups) or with Wilcoxon signed rank test (comparison before/after within a treatment).

Correlations between some of the variables were done by using the Spearman rank correlation test.

Study Patients

Disposition of Subjects

70 subjects were included in the study (Table 2). One of the subjects terminated the study before consumption of any study product and four of the excluded subjects did not return their diaries and therefore safety could not be identified for these subjects. 65 subjects were included in the safety set and 63 subjects were included in the FAS. Among the seven excluded from FAS, three of the subjects consumed antibiotics between the time of inclusion and the TSST challenge, two of the subjects could not come on the test day and one of the subjects reported an adverse event and did not want to continue. Five of the subjects were excluded due to consumption of study product more than 33 days and one subject was excluded due to non-comparable TSST, leaving 57 subjects in the PPS.

TABLE 2
Number of subjects included in the different data sets.

| Total number | | | |
|---|---|---|---|
| of subjects | Safety set | Full Analysis Set | Per-protocol Set |
| 70 | 65 | 63 | 57 |

Results

Demographic and Baseline Data

The FAS population was composed of 63 subjects with a mean age of 23.5 years and a mean BMI of 23.4 (Table 3). The PP population consisted of 57 subjects (5 subjects were excluded due to an intervention period of over 33 days and one due to a delay of the TSST test for about 1 hour).

TABLE 3
Summary of demographics and other baseline
characteristics (mean, range)

| | FAS | | PP | |
|---|---|---|---|---|
| | L. plantarum | FAS | L. plantarum | PP |
| | DSM 15312 | Placebo | DSM 15312 | Placebo |
| | (n = 32) | (n = 31) | (n = 30) | (n = 27) |
| Females | 63% | 71% | 63% | 70% |
| Age, year | 25.3 (19-35) | 24.3 (19-32) | 25.4 (19-35) | 23.8 (19-32) |
| BMI | 22.4 (19-29) | 21.9 (18-26) | 22.2 (19-29) | 21.9 (18-26) |

Prior Consumption of Probiotics

Few subjects consumed probiotics prior to Visit 1 (4 of 70, 6%).

Treatment Compliance

Intake of study product was recorded daily by the subjects in the diary. Additionally, the number of capsules dispensed and returned by each subject was documented at Visit 2. A summary of the compliance is presented in Table 4. The compliance was good, ranging from 92 to 107% and thus no subject had to be excluded from the per protocol analysis due to a compliance lower than 80%.

TABLE 4
Summary of compliance (%, FAS)^{a}

| | FAS LPHEAL9 | FAS Placebo | Totally |
|---|---|---|---|
| | (n = 32) | (n = 31) | (n = 63) |
| Mean (SD) | 98.6 (2.9) | 99.2 (2.9) | 98.9 (2.9) |
| Median | 100 | 100 | 100 |
| Min-max | 92.3-103.3 | 93.3-107.1 | 92.3-107.1 |

^{a}Calculated as (Number of capsules taken/Number of days between Visit 1 and Visit 2) * 100.

Counts of Lactobacilli in the Saliva

At the start, before intervention, 76% of the subjects had detectable levels of lactobacilli in the saliva. The counts of lactobacilli in saliva increased significantly in the group that consumed LPHEAL9, from a median value of 7.3 log 10 cfu/ml before the intervention to 9.3 log 10 cfu/ml after the intervention (Table 5). After four weeks' intake, the level of lactobacilli in saliva was significantly higher in this group compared to the placebo group.

TABLE 5
Lactobacilli counts (log₁₀ cfu/ml) in the saliva before and after
the intervention (median, min-max, FAS)

| | | | | P-value^{a} |
|---|---|---|---|---|
| | Before | After 4 weeks | | Within |
| | intervention | intervention | Change | group |
| LPHEAL9 | 7.3 | 9.3 | 2.1 | 0.000 |
| (n = 32) | (<2.9-12.9) | (<2.9-17.6) | (−3.1-012.1) | |
| Placebo | 4.6 | 5.3 | 0 | 0.741 |
| (n = 31) | (<2.9-12.2) | (<2.9-13.4) | (−3.6-3.7) | |
| P-value^{b} | 0.274 | 0.000 | 0.000 | |
| Between groups | | | | |

^{a}Wilcoxon signed rank test. 2-sided.
^{b}Wilcoxon rank sum test. 2-sided.

SMBQ Global score and State-Trait Anxiety Inventory (STAI-S)

At inclusion all subjects had to be classified as highly stressed according to the SMBQ questionnaire (global score 3.75). After four weeks intervention, the SMBQ global score was reduced significantly in both groups but no significant difference between the groups was found at any time point (Table 6). About one third of the subjects had a SMBQ global score below 3.75 after the intervention, and thus were not classified as highly stressed any longer. The global scores for SMBQ before the intervention were about the same as had earlier been observed in a TSST study on highly stressed subjects (mean value 4.64, Linninge et al, 2018, Biol Psychol, 138:48-55).

TABLE 6
SMBQ global score before and after the intervention (mean, SD)

| | | After | | P-value^{a} |
|---|---|---|---|---|
| | Before | four weeks' | | Within |
| | intervention | intervention | Change | group |
| L. plantarum | 4.79 | 4.13 | −0.65 | 0.0000 |
| DSM 15312 | (0.63) | (0.76) | (0.76) | |
| (n = 32) | | | | |
| Placebo | 4.66 | 4.18 | −0.49 | 0.0002 |
| (n = 31) | (0.62) | (0.90) | (0.66) | |
| P-value^{b} | 0.4623 | 0.7249 | 0.3255 | |
| Between groups | | | | |

^{a}Wilcoxon signed rank test. 2-sided.
^{b}Wilcoxon rank sum test. 2-sided.

Both the L. plantarum DSM 15312 (HEAL 9™) group and the placebo group reported an induced acute stress effect, measured by the State-Trait Anxiety Inventory (STAI-S), related to the TSST test (p<0.0001). The scores for STAI-S before and after the test (40 and 54, respectively) were about the same as had been observed earlier in a TSST study on highly stressed subjects (Linninge et al, 2018, supra).

Efficacy Results

Primary Efficacy Analysis—Serum Cortisol Levels

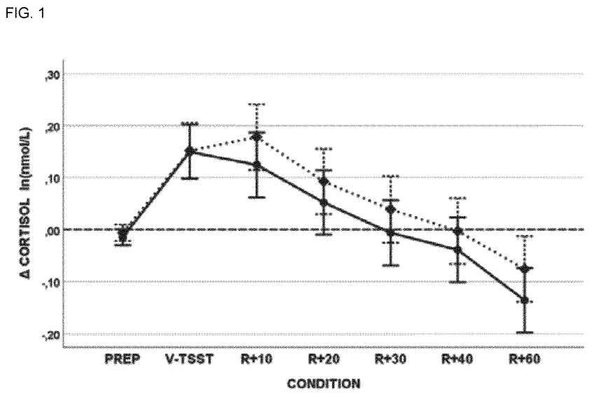

A main effect of CONDITION (i.e. time point), F(6, 342)=21.42, p<0.0001, η²=0.27, ε=0.409, showed that cortisol increased after stress induction and after 10 min of recovery, and then slowly decreased as a function of time, linear contrast Fₗᵢₙₑₐᵣ(1, 57)=18.72, p<0.0001, η²=0.25, quadratic contrast F_{quad}(1, 57)=24.35, p<0.0001, η²=0.30, and cubic contrast F_{cubic}(1, 57)=23.10, p<0.0001, η²=0.29, see FIG. 1. No other significant effects were found.

Post hoc analysis of PP subjects with a higher cortisol level after the test than before the test (77% of the subjects, LPHEAL9 n=23, PLACEBO n=21) showed a significant increase in cortisol levels compared to baseline, 0 and 10 minutes after the TSST test, for both groups. For the placebo group was it also significantly increased compared to before the test at 20 and 30 minutes. There was no significant difference between groups at any time point but at 10 minutes after the test there was a trend for a lower cortisol level in the LPHEAL9 group compared to the PLACEBO group (p=0.07).

Secondary Efficacy Analyses—Inflammation Markers and Zonulin

Soluble Fractalkine in Plasma

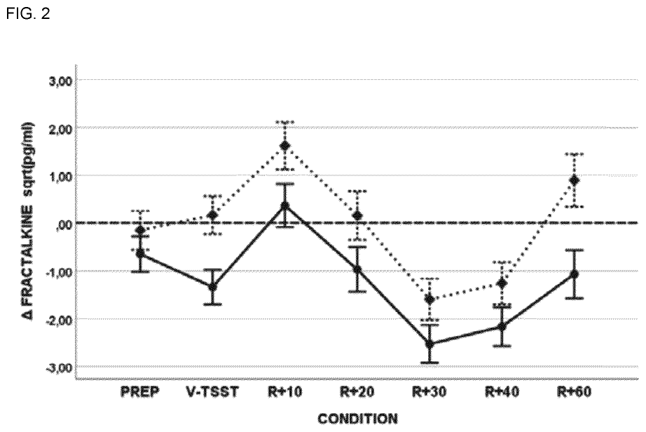

Fractalkine increased and peaked 10 min after V-TSST and then gradually decreased during the following 20 min (see FIG. 2). After that, fractalkine increased again during the remaining recovery, F(6, 318)=14.21, p<0.0001, η²=0.21, E=0.737, Fₗᵢₙₑₐᵣ(1, 53)=7.12, p<0.010, η²=0.12, and F_{cubic}(1, 53)=39.61, p<0.0001, η²=0.43.

A main effect of GROUP (i.e. treatment group) was found, F(1, 53)=9.41, p=0.003, η²=0.15. Compared to baseline the PLACEBO group's fractalkine levels increased more, and remained above, the fractalkine levels of the LPHEAL9 group. The CONDITION*GROUP interaction effect was not significant.

The group effect seen was also confirmed when single time points were compared. The fractalkine level was significantly lower in the LPHEAL19 group than in the PLACEBO group, 20 and 60 minutes after the test.

Soluble CD163 (sCD163) in Plasma

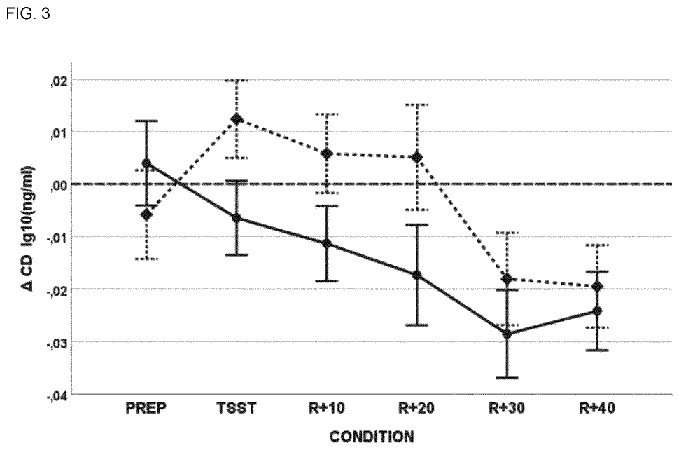

A main effect of CONDITION generally showed that sCD163 values decreased from preparation to the end of the 40 min recovery, F(5, 295)=10.93, p<0.0001, η²=0.16, E=0.898, and Fₗᵢₙₑₐᵣ(1, 59)=38.08, p<0.0001, η²=0.39 (see FIG. 3).

Also the GROUP*CONDITION was significant, F(5, 295)=3.07, p=0.013, η2=0.05, ε=0.898, F_{quad}(1, 59)=12.87, p<0.001, η2=0.18. After baseline the placebo group's sCD163 values decreased at preparation and then increased and peaked after stress induction. After that, the values gradually decreased as a function of time. The sCD163 values for the active group increased during preparation and then decreased during recovery.

Comparing single time points showed that the sCD163 level was significantly lower in the LPHEAL19 group than in the PLACEBO group 0, 10, 20 and 60 minutes after the test.

The result for sCD163 confirms the result for fractalkine since sCD163 and fractalkine are both released by ADAM-17 protease.

Plasma IFN-γ

After an initial small decrease IFN-γ increased and reached a maximum at 10 min after stress induction, after which it decreased below baseline levels during the last 30 min of recovery, F(6, 348)=10.66, p<0.0001, η²=0.16, E=0.702, F_{quad}(1, 58)=4.87, p=0.031, η²=0.08, and F_{cubic}(1, 58)=41.40, p<0.0001, η²=0.42. No other significant effects were found.

Plasma IL-1Beta

The level of IL-1beta did not change significantly and no significant difference between the groups was found.

Plasma IL-10

No significant effects were found.

Plasma IL-6

The results showed a significant main effect of CONDITION, F(6, 330)=86.74, p<0.0001, η²=0.61, E=0.502, Fₗᵢₙₑₐᵣ(1, 55)=173.70, p<0.0001, η²=0.76, and F_{cubic}(1, 55)=19.77, p<0.0001, η²=0.26. After preparation, IL-6 starts to increase rather steeply. After 10 min of recovery it continuous to increase, but less steeply, until 40 min of recovery, after which it increased more steeply again. No other significant effects were found.

Plasma IL-8

A main effect of CONDITION was found, F(6, 354)=9.92, p<0.0001, η²=0.14, E=0.831. During the preparation condition IL-8 levels were slightly lower than during baseline. Then IL-8 levels increased at V-TSST and then decreased until 30 min of recovery, followed by a second increase after 40 min of recovery, F_{quad} (1, 59)=14.79, p<0.001, η²=0.20, and F_{cubic}(1, 59)=16.35, p<0.0001, η²=0.22.

Also the main effect of GROUP was significant, F(1, 59)=4.26, p=0.043, η²=0.07. The IL-8 levels in the LPHEAL19 group increased more than those of the PLACEBO group, and remained higher during the rest of recovery. The CONDITION*GROUP interaction effect was not significant.

Plasma TNF-α

The main effect of CONDITION was significant, F(6, 348)=18.38, p<0.0001, η²=0.24, ε=0.819. After a small decrease at preparation and V-TSST, TNF-α levels increased and peaked after 10 min of recovery. Then the levels gradually decreased until 40 min of recovery when a rapid increase occurred, Fₗᵢₙₑₐᵣ(1, 58)=8.32, p=0.005, η²=0.13, F_{quad}(1, 58)=7.96, p=0.007, η²=0.12, and F_{qubic}(1, 58)=64.61, p<0.0001, η²=0.53. No other significant effects were found.

Serum Zonulin

No significant effects were found.

Secondary Efficacy Analyses—Pulse and Heart Rate Variability

Pulse Rate (HR)

The main effect of CONDITION was significant, F(6, 330)=107.49, p<0.0001, η²=0.66, E=0.44, Fₗᵢₙₑₐᵣ=(1, 55)=157.37, p<0.0001, η²=0.74, F_{quad}n(1, 55)=18.94, p<0.0001, η²=0.26, F_{cubic}(1, 55)=115.22, p<0.0001, η²=0.68. After baseline, HR increased and peaked at SPEECH, after which it decreased and remained at about baseline levels.

Heart Rate Variability (HF-HRV)

HF-HRV decreased during PREP, SPEECH and MATH, and then increased during recovery the first 10 min. Then it slightly decreased during the rest of recovery. F(3, 330)=6.12, p=0.001, η²=0.10, ε=0.46, Fₗᵢₙₑₐᵣ(1, 55)=8.09, p=0.006, η2=0.13, and F_{quad}(1, 55)=13.32, p=0.001, η²=0.20.

Secondary Efficacy Analyses—Gut Function

Abdominal pain, flatulence and bloating were evaluated three times: at inclusion before intake of study product, at the end of the intervention period before the TSST test, and after the TSST test. No significant differences between the groups in gut function were found.

Safety Evaluation

Gastrointestinal adverse events were reported by 21 subjects (32%). Most of the events were mild and possibly related to intake of the study product (Table 7). There were no differences between the groups in reported gastrointestinal adverse events; in the LPHEAL9 group reported 9 subjects 16 events and in the placebo group reported 12 subjects 20 events The most common gastrointestinal adverse event was abdominal pain (8 events), followed by bloating (7 events) and rumbling stomach and nausea (5 events each).

One subject in the placebo group had a stress related serious adverse event, just after the TSST test had finished. The subject fainted, fell and the shoulder was dislocated. The subject had to go to the hospital for help to get the shoulder back in place. The subject experienced this also one month before but did not report this to the study staff during inclusion. The event was unlikely related to the intake of study product.

TABLE 7
Reported gastrointestinal adverse events during the study (safety set)

| | LPHEAL9 | Placebo | Total |
|---|---|---|---|
| | N = 33 | N = 32 | N = 65 |
| Reported AE (N) | 16 | 20 | 36 |
| Intensity | | | |
| Mild | 14 | 17 | 31 |
| Moderate | 2 | 3 | 5 |
| Severe | | | |
| Causality | | | |
| Unlikely related | | | |
| Possibly related | 16 | 18 | 34 |
| Probably related | 0 | 2 | 2 |
| Outcome | | | |
| Resolved | 16 | 19 | 35 |
| Continued | 0 | 1 | 1 |
| Description of AE | | | |
| Abdominal pain | 3 | 5 | 8 |
| Bloating | 2 | 5 | 7 |
| Rumbling stomach | 2 | 3 | 5 |
| Nausea | 3 | 2 | 5 |
| Flatulence | 2 | 2 | 4 |
| Diarrhea | 3 | 0 | 3 |
| Constipation | 1 | 1 | 2 |
| Hemorrhoids | 0 | 1 | 1 |
| Herpes simplex | 0 | 1 | 1 |

Discussion and Overall Conclusions

In individuals with chronic stress leading up to the trial, the episode of acute psychosocial stress caused by the TSST resulted in a release of cortisol that was higher and longer for the placebo group compared to the Lactobacillus strain treated group. Hence, pre-treatment with the Lactobacillus strain (L. plantarum DSM 15312) reduced cortisol release due to acute psychosocial stress, compared to placebo control.

For the first time, it has been determined that an episode of acute psychosocial stress causes a release of fractalkine. In individuals with chronic stress subjected to an episode of acute psychosocial stress (the TSST), release of fractalkine was observed at 10 minutes in the recovery phase, followed by a further release observed at 60 minutes in the recovery phase. Pre-treatment with the Lactobacillus strain (L. plantarum DSM 15312) caused a significant reduction in fractalkine levels (release of soluble fractalkine) compared to placebo control.

In individuals with chronic stress, the episode of acute psychosocial stress caused by the TSST resulted in increased plasma levels of soluble CD163 compared to baseline in the placebo control group. However, pre-treatment with the Lactobacillus strain (L. plantarum DSM 15312) prevented this increase during and after the test. Hence, the results for sCD163 appear to confirm the effect of the Lactobacillus strain (L. plantarum DSM 15312) on the acute psychosocial stress-induced release of fractalkine since both fractalkine and sCD163 are released by ADAM17 protease.

Therefore, Lactobacillus administration, particularly Lactobacillus plantarum DSM 15312 (HEAL 9™) is useful for the reduction and/or prevention of at least one deleterious effect of acute psychosocial stress in a human, particularly elevated levels of cortisol, soluble fractalkine, and soluble CD163 in plasma.

### Experimental Example 2

Introduction

To determine whether the positive effects of Lactobacillus treatment observed in Experimental Example 1 were limited to acute psychosocial stress in the context of chronic stress, or whether they could be expected to benefit all individuals experiencing acute psychosocial stress, we performed further analysis of the data from individuals in Experimental Example 1 who were not defined as having chronic stress on the day of the TSST test.

Methods

All details of the methods are according to Experimental Example 1. However, in Experimental Example 1 a mixture of subjects that were low or high stressed on the day of the TSST test were included. Experimental Example 2 includes a separate analysis of the individuals in example 1 that had a SMBQ score of 3.75 or greater on the day of the TSST test (such individuals are designated as “high stressed” [HS] subjects) and the individuals in Example 1 that had a SMBQ score of less than 3.75 on the day of the TSST test (such individuals are designated as “low stressed” [LS] subjects).

Results

On the test day, 9 subjects in the active (L. plantarum DSM 15312 [HEAL 9™]) group and 12 subjects in the placebo group had an SMBQ score of less than 3.75 and were designated as LS subjects.

The levels of fractalkine, soluble CD163 and cortisol in HS and LS subjects only are shown in Tables 8-10 below.

Soluble Fractalkine in Plasma

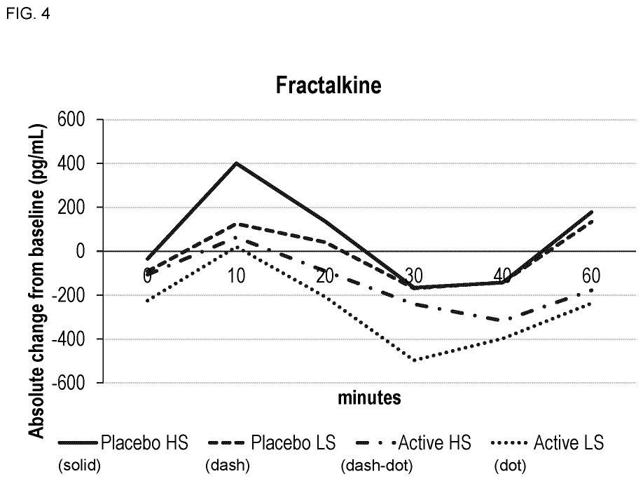

There was no significant difference at any time point between chronically stressed subjects (SMBQ value of ≥3.75; n=42) and LS subjects (SMBQ value of <3.75; n=21) (treatment groups merged). The fractalkine level was significantly lower for LS subjects that had consumed LP HEAL9 compared to the corresponding subjects in the placebo group, 30 minutes after the test. See FIG. 4 and Table 8.

TABLE 8
Fractalkine levels (pg/mL) in plasma after the TSST test
(mean change from before the test); HS/LS subjects

| Time after | LP HEAL9 | Placebo | P-value¹, |
|---|---|---|---|
| TSST test | group | group | between groups |
| 0 min | −107/−225 | −35/−91.7 | 0.079/0.193 |
| +10 min | 63/20 | 400/125 | 0.397/0.247 |
| +20 min | −91/−208 | 136/41 | 0.126/0.201 |
| +30 min | −241/−497 | −166/−169 | 0.487/0.028 |
| +40 min | −317/−398 | −143/−142 | 0.383/0.227 |
| +60 min | −178/−237 | 178/134 | 0.060/0.227 |

¹Wilcoxon rank sum test

Soluble CD163 in Plasma

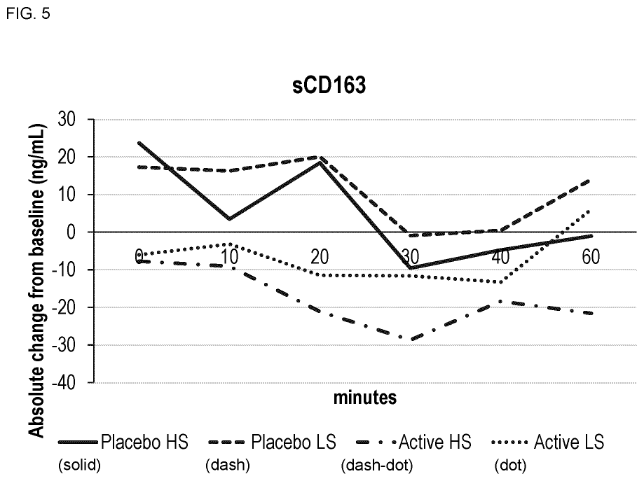

There was a significant difference at the last timepoint (p=0.021) between chronically stressed subjects (SMBQ value of ≥3.75) and LS subjects (SMBQ value of <3.75) (−12.30 and 10.81, respectively) (treatment groups merged) The sCD163 level was significantly lower for LS subjects that had consumed LP HEAL9 compared to the corresponding subjects in the placebo group, 0 and 10 minutes after the test. See FIG. 5 and Table 9.

TABLE 9
Soluble CD163 levels (ng/mL) in plasma after the TSST test
(mean change from before the test); HS/LS subjects.

| Time after | LP HEAL9 | Placebo | P-value¹, |
|---|---|---|---|
| TSST test | group | group | between groups |
| 0 min | −7.73/−6.05 | 23.69/17.28 | 0.010/0.028 |
| +10 min | −9.10/−3.17 | 3.46/16.28 | 0.161/0.025 |
| +20 min | −21.14/−11.50 | 18.42/20.04 | 0.103/0.076 |
| +30 min | −28.69/−11.64 | −9.59/−0.93 | 0.397/0.356 |
| +40 min | −18.40/−13.30 | −4.76/0.46 | 0.604/0.155 |
| +60 min | −21.60/6.04 | −1.05/14.00 | 0.079/0.322 |

¹Wilcoxon rank sum test

Serum Cortisol

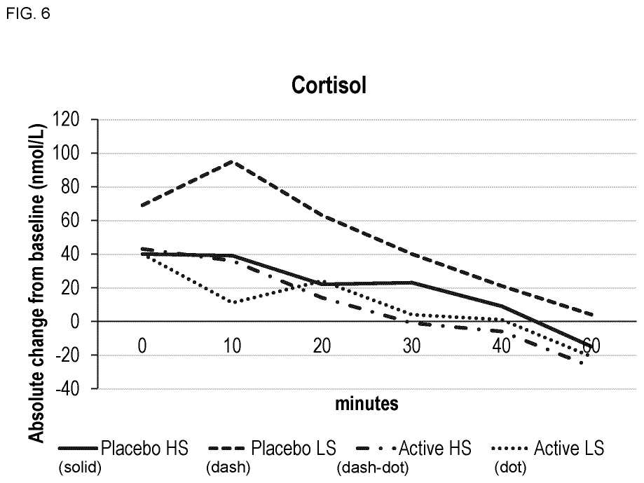

There was no significant difference at any time point between chronically stressed subjects (SMBQ value of 3.75; n=42) and LS subjects (SMBQ value of <3.75; n=21) (treatment groups merged). For LS subjects there was a significant difference in cortisol between the LP HEAL9 and the placebo groups 10 minutes after the test (p=0.025). See FIG. 6 and Table 10.

TABLE 10
Cortisol levels (pg/mL) in serum after the TSST test
(mean change from beforethe test); HS/LS subjects.

| Time after | LP HEAL9 | Placebo | P-value¹, |
|---|---|---|---|
| TSST test | group | group | between groups |
| 0 min | 43/40 | 40/69 | 0.343/0.241 |
| +10 min | 36/11 | 39/94 | 0.649/0.025 |
| +20 min | 14/24 | 22/63 | 0.889/0.277 |
| +30 min | −1/4 | 23/40 | 0.854/0.058 |
| +40 min | −6/1 | 9/21 | 0.940/0.277 |
| +60 min | −27/−20 | −15/4 | 0.714/0.219 |

¹Wilcoxon rank sum test

1. A method for reducing and/or preventing at least one deleterious effect of acute psychosocial stress in a human, wherein the method comprises treating by administering to a human an effective dose of at least one strain of Lactobacillus, and wherein the deleterious effect is an elevated level of soluble fractalkine.

2. A method for reducing and/or preventing at least one deleterious effect of acute psychosocial stress in a human, wherein the method comprises treating by administering to a human an effective dose of at least one strain of Lactobacillus, wherein the at least one deleterious effect is an elevated level of soluble fractalkine and at least one further deleterious effect of acute psychosocial stress.

3. The method according to claim 2, wherein the at least one further deleterious effect of acute psychosocial stress is selected from one or more of a biochemical and/or a physiological indicator of stress.

4. The method according to claim 3, wherein the biochemical indicator is selected from an elevated level of one or more cytokines, especially inflammatory cytokines and preferably one or more of cortisol and/or soluble CD163.

5. The method according to any of claims 2 to 4, wherein the further deleterious effect is an elevated level of soluble CD163.

6. The method according to any preceding claim, wherein the elevated level of soluble fractalkine is an elevated plasma level of soluble fractalkine.

7. The method according to any preceding claim, wherein the human to be treated has chronic stress.

8. The method according to claim 7, wherein chronic stress is indicated by a score of 3.75 or greater in the Shirom-Melamed Burnout Questionnaire.

9. The method according to any preceding claim, wherein the effective dose of the at least one strain of Lactobacillus is from about 1×10⁶ to about 1×10¹⁴ colony forming units (CFU) per dose.

10. The method according to claim 9, wherein the effective dose of the at least one strain of Lactobacillus is from about 1×10⁸ to about 1×10¹² CFU per dose.

11. The method according to claim 10, wherein the effective dose of the at least one strain of Lactobacillus is from about 1×10⁹ to about 1×10¹¹ CFU per dose.

12. The method according to claim 11, wherein the effective dose of the at least one strain of Lactobacillus is 1×10¹⁰ CFU per dose.

13. The method according to any preceding claim, wherein the effective dose of the at least one strain of Lactobacillus is administered at least once a day.

14. The method according to any preceding claim, wherein the effective dose is administered daily for at least one week.

15. The method according to claim 14, wherein the effective dose is administered daily for at least four weeks.

16. The method according to any preceding claim, wherein the at least one strain of Lactobacillus is selected from the species Lactobacillus plantarum, Lactobacillus paracasei, Lactobacillus rhamnosus, Lactobacillus crispatus, Lactobacillus gasseri, Lactobacillus fermentum, Lactobacillus reuteri, Lactobacillus acidophilus, Lactobacillus helveticus, Lactobacillus casei, Lactobacillus salivarius, and Lactobacillus johnsonii.

17. The method according to claim 16, wherein the at least one strain of Lactobacillus plantarum is selected from Lactobacillus plantarum DSM 15312, Lactobacillus plantarum DSM 15313, Lactobacillus plantarum DSM 15316, Lactobacillus plantarum DSM 6595, Lactobacillus plantarum DSM 9843, Lactobacillus plantarum DSM 32131, Lactobacillus plantarum DSM 17852, and Lactobacillus plantarum DSM 17853.

18. The method according to claim 16 or 17, wherein the at least one strain of Lactobacillus plantarum is able to adhere to the intestinal epithelium and persist in the intestine.

19. The method according to any one of claims 16 to 18, wherein the at least one strain of Lactobacillus plantarum comprises a mannose-specific adhesin.

20. The method according to any one of claims 16 to 19, wherein the at least one strain of Lactobacillus plantarum is Lactobacillus plantarum DSM 15312 (HEAL 9™).

21. The method according to any preceding claim, wherein the at least one strain of Lactobacillus is administered in the form of a composition comprising at least one carrier, excipient and/or diluent material.

22. The method according to claim 21, wherein the composition is provided in the form of a solution, suspension, emulsion, tablet, granule, powder, capsule, lozenge, chewing gum, or suppository.

23. The method according to claim 22, wherein the composition is provided in the form of capsule.

24. The method according to claim 21, wherein the carrier material is a food.

25. At least one strain of Lactobacillus for use in a method of reducing and/or preventing at least one deleterious effect of acute psychosocial stress in a human, wherein the method comprises treatment by administration of an effective dose of the at least one strain of Lactobacillus, and wherein the deleterious effect is an elevated level of soluble fractalkine.

26. At least one strain of Lactobacillus for use in a method of reducing and/or preventing at least one deleterious effect of acute psychosocial stress in a human, wherein the method comprises treatment by administration of an effective dose of the at least one strain of Lactobacillus, and wherein the at least one deleterious effect is an elevated level of soluble fractalkine and at least one further deleterious effect of acute psychosocial stress.

27. At least one strain of Lactobacillus for use according to claim 25, wherein the at least one further deleterious effect of acute psychosocial stress is selected from one or more of a biochemical and/or a physiological indicator of stress, optionally wherein the biochemical indicator is selected from an elevated level of one or more cytokines, especially inflammatory cytokines and preferably is an elevated level of soluble CD163 or cortisol.

28. A composition as defined in claim 21, for use in reducing and/or preventing at least one deleterious effect of acute psychosocial stress in a human.

29. A method as claimed in any one of claims 1-24, for use in a method of treating, preventing and/or reducing at least one symptom of cancer, inflammatory disease, a cardiovascular disease, inflammatory bowel disease, irritable bowel syndrome (IBS), ulcerative colitis and/or Crohn's disease in a human.

30. At least one probiotic strain of Lactobacillus for use as claimed in any of claims 25 to 27, wherein the use is in a method of treating, preventing and/or reducing at least one symptom of cancer, chemotherapy-induced peripheral neuropathy (CIPN), diabetic retinopathy (DR), inflammatory disease, a cardiovascular disease, inflammatory bowel disease, irritable bowel syndrome (IBS), ulcerative colitis and/or Crohn's disease in a human.

31. A composition for use as claimed in claim 28, wherein the use is in a method of treating, preventing and/or reducing at least one symptom of cancer, chemotherapy-induced peripheral neuropathy (CIPN), diabetic retinopathy (DR), inflammatory disease, a cardiovascular disease, inflammatory bowel disease, irritable bowel syndrome (IBS), ulcerative colitis and/or Crohn's disease in a human.
